(19) United States
(12) Patent Application Publication
(10) Pub. No.: US 2017/0275683 A1
(43) Pub. Date: Sep. 28, 2017

(54) Method for High-Throughput, Ultra Long-Read DNA Sequencing

(71) Applicant: Allen Bruce Futcher, Setauket, NY (US)

(72) Inventor: Allen Bruce Futcher, Setauket, NY (US)

(21) Appl. No.: 14/923,356

(22) Filed: Oct. 26, 2015

(51) Int. Cl. C12Q 1/68

(52) U.S. Cl. CPC C12Q 1/6853; C12Q 1/6809; C12Q 1/6806; C12Q 1/6811; C12Q 1/6841; C12Q 1/6846; C12Q 1/68; C12Q 2600/16; C12Q 2525/161

(57) ABSTRACT

The Invention is a method for ascertaining extremely long DNA sequence reads (kilobases or megabases) from polony-type DNA sequencers. Polony-type DNA sequencers (e.g., Illumina, Roche, and Life Technologies sequencers) typically give read lengths of only about 500 bp. The Invention can extend those read lengths by orders of magnitude.

### CROSS-REFERENCES TO RELATED APPLICATIONS

A provisional patent application covering this Invention has previously been filed, with the title “Long Read Sequencing after DNA Combing”, and the Ser. No. 62/069,359, with the deadline of Oct. 28, 2015 for conversion into a utility application. While the title for this non-provisional application is slightly different, the invention is exactly the same.

### STATEMENT REGARDING FEDERALLY SPONSORED RESEARCH OR DEVELOPMENT

Not applicable.

### TECHNICAL FIELD

The invention is a method for achieving high-throughput, ultra-long DNA sequence reads on DNA sequencers that, generally, use amplified DNA molecules (emulsion PCR or bridge amplification) rather than single molecules as templates. Such DNA sequencers ordinarily produce only short reads (about 500 bases), and include sequencers manufactured by Illumina, Roche, and Life Technologies.

### BACKGROUND OF THE INVENTION

Approximately 15 years ago, “Next Generation” or “High Throughput” DNA sequencers were developed. These typically read only short contiguous sequences of DNA (about 500 bases), but do so on a very large scale: tens or hundreds of millions of individual reads. The total data output from a run of a DNA sequencer can be calculated by multiplying the read length by the number of reads, and for a large DNA sequencer, this output can be 600 gigabases or more. These sequencers ascertain sequence from a “polony” (“PCR colony”, a phrase coined by George Church) (https://en.wikipedia.org/wiki/Polony_%28biology%29) of many amplified and localized molecules originating in a single molecule. For several hundred bases, the sequences of the individual molecules in the polony can be read in synchrony, yielding interpretable data. But inevitably, synchrony degrades at around 500 bases, and interpretable data can no longer be obtained. Such sequencers will be defined as and referred to here as “polony” sequencers. There is a very large literature describing the properties of such sequencers (e.g., Nguyen and Burnett, “Automation of molecular-based analyses: a primer on massively parallel.sequencing.” Clin. Biochem. Rev. 2014, vol 35, 169-76.), and a great deal of information is available at the websites of Illumina, Life Technologies, etc. (https://www.illumina.com/, http://www.thermofisher.com/us/en/home/life-science/sequencing.html)

Even more recently, other kinds of DNA sequencers have been developed which, instead of reading from a polony of amplified DNA, read a single molecule of DNA. For instance, such sequencers are made by Pacific Biosciences (http://www.pacb.com/) and by Oxford Nanopore (https://www.nanoporetech.com/). Compared to polony sequencers, these single-molecule sequencers have the great advantage that (since only one molecule is being read and therefore no synchrony is involved) read lengths can be very long—many kilobases. However, they also have two disadvantages. First, because the signal coming from a single molecule is inevitably weak, these sequencers have a high error rate. Second, and perhaps even more serious, the number of molecules addressable by these sequencers is much smaller than the number of molecules addressable by the “polony” sequencers, and so the total data output per run is much smaller, even though the read length is longer.

In many applications, the relatively short read length of the polony sequencers is a serious disadvantage. One important example expounded here is the problem of determining human haplotypes. Humans are diploids, and so have two copies of each chromosomes (excepting, for males, the X and Y chromosomes), with one copy inherited from each parent. In general, outside of regions of recombination, a very long region of one chromosome is entirely inherited from the father, and the corresponding region of the other chromosome is inherited from the mother. A short-read polony sequencer cannot associate any part of any such paternal or maternal region with any other part of the same paternal or maternal region. This is crucial in the diagnosis of some genetic diseases. For instance, consider a woman concerned about the status of her BRCA1 gene, which, when mutant, causes high rates of breast cancer. A polony-sequencer might reveal two different crippling mutations in the woman's BRCA1 gene. But the BRCA1 gene is extremely long. Are the two crippling mutations both in the same gene (e.g., both in the gene inherited from the father)? In this case, the woman still has one entirely wild-type (fully functional) gene, inherited from the mother, and is not at high risk of breast cancer. But, on the other hand, if one crippling mutation is in the paternal gene, and the other crippling mutation is in the maternal gene, then the woman has no functional copy of BRCA1, and is at high risk. This situation is difficult to diagnose at present, and the Invention here would provide a straightforward way for such diagnosis, because the long sequences produced by the Invention would directly say whether the two crippling mutations are on the same molecule, or on different molecules.

### BRIEF SUMMARY OF THE INVENTION

The Invention is a method for ascertaining the equivalent of extremely long reads (kilobases, tens of kilobases, or more) from polony-type sequencers, especially those with a planar flow cell such as the Illumina sequencers. A polony sequencer using the Invention would have the advantage of extremely long effective reads, while retaining the advantages of low error rate and high-throughput, thus combining the advantages of the two present types of high-throughput sequencers. Furthermore, the invention can be applied to existing polony sequencers, of which there are thousands in use.

The approach is to stretch very long single molecules of DNA out upon the flow cell, and have them bind the surface of the flow cell. These long molecules are then fragmented and amplified in situ, such that the amplified polonies from a single original molecule are now in line with one another. Sequencing at each polony occurs. Finally, image and sequence analysis software is used to deconvolute the many polonies on the flow cell, assigning particular polonies to the same original long DNA molecule, and allowing reconstruction of a long region of DNA sequence. Note that these sequences may be gapped and non-contiguous, but that the same process applied to other instances of the same region of DNA will fill in any gaps, ultimately generating continuous ultra-long sequence information.

### BRIEF DESCRIPTION OF THE DRAWINGS:

FIG. 1, Current Illumina Sequencing. A simple drawing illustrating polonies on the surface of an Illumina flow cell, as used currently for high-throughput sequencing.

FIG. 2, DNA Combing. A part of the Invention, illustrating two long combed DNA molecules being stretched out upon the surface of a flow cell.

FIG. 3, Tagmentation. A part of the Invention, illustrating polonies arising in two lines from the two molecules of FIG. 2, after tagmentation, amplification, and polony formation.

FIG. 4, Reconstruction. A part of the Invention, illustrating how gaps in a single line of polonies can be filled using polonies from other instances of the same molecule.

### DETAILED DESCRIPTION OF THE INVENTION:

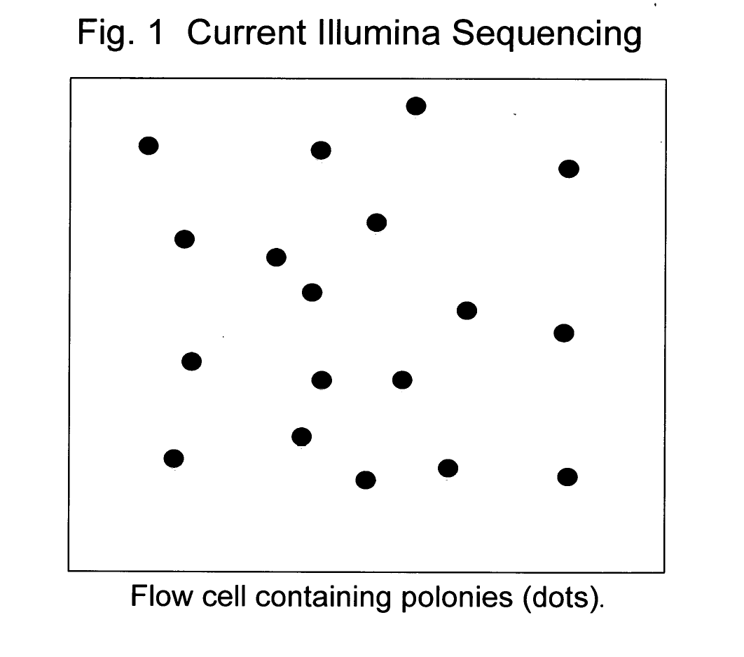

Although there are many embodiments of the invention, the most obvious is the embodiment on an Illumina flow cell, a planar piece of modified glass with attached oligonucleotides. The description below refers to this Illumina flow cell embodiment (FIG. 1).

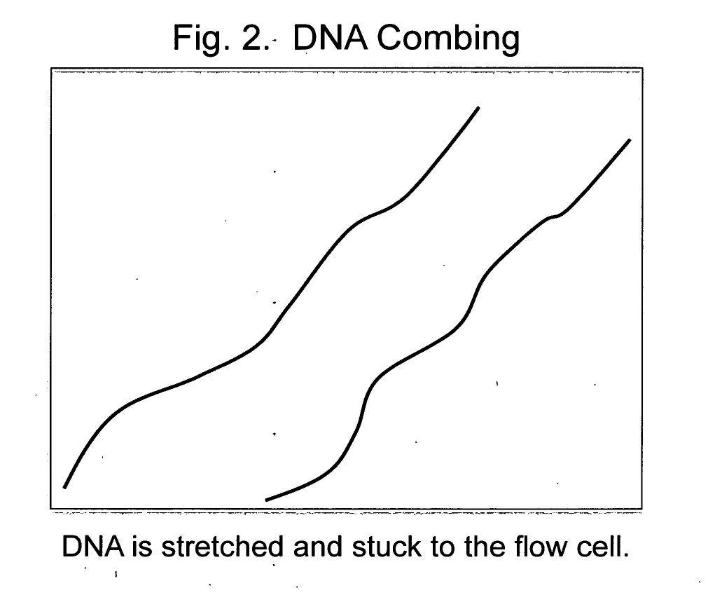

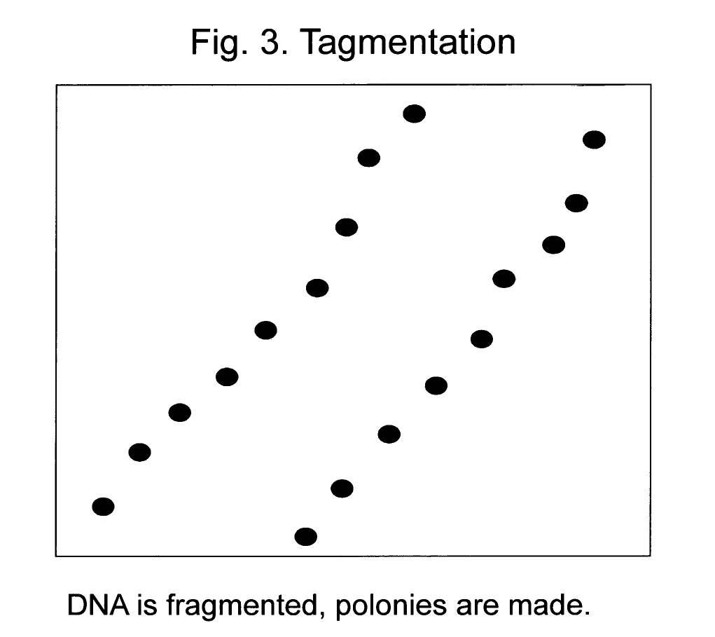

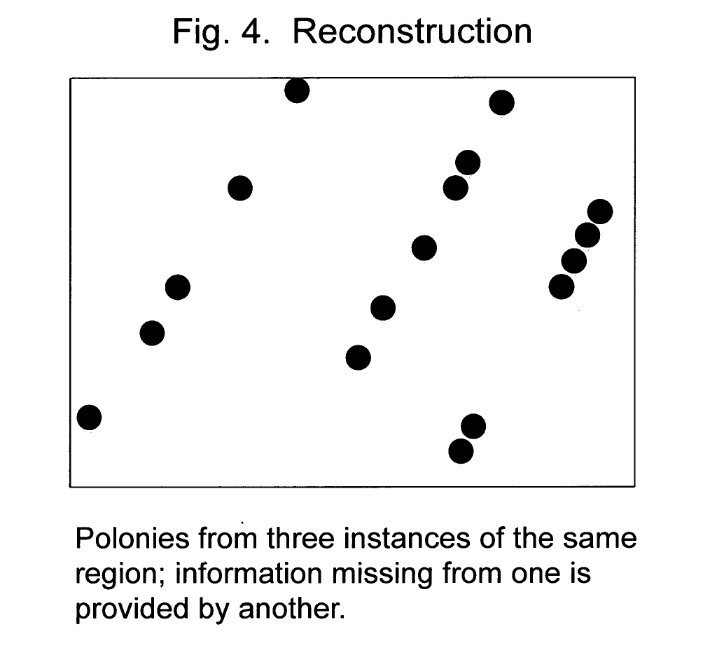

None of the individual steps below are entirely novel. DNA combing (step 1) (FIG. 2), DNA binding to substrates (step 2), tagmentation (step 3) (FIG. 3), sequencing (step 4), image-based sequencing of polonies (step 5), and image- and sequence-based sequence reconstruction (step 6) (FIG. 4) are already individually understood. The invention is unique and novel in the sequential application of these six methods to yield the result of extremely long sequence reads on a polony-based sequencer.

1. The procedure begins with long (tens of kilobases or megabase) DNA molecules. These are applied to the flow cell in solution, and stretched over the flow cell by some embodiment of DNA combing (FIG. 2). For instance, one part of a DNA molecule, preferably one end, might be attached to the flow cell. (Attaching both ends would also work.) Then (a) an electric current; or (b) fluid flow; or (c) any other method of DNA combing would be used to stretch the DNA molecules in a particular direction. DNA combing is well studied, and has many embodiments, many of which are potentially applicable here (Bianco et al., 2012, “Analysis of DNA replication profiles in budding yeast and mammalian cells using DNA combing.”, Methods 57(2):149-57; Herrick and Bensimon, 2009, “Introduction to molecular combing: genomics, DNA replication, and cancer.” Methods Mol. Biol. 521:71-101; Lebofsky and Bensimon, 2003, “Single DNA molecule analysis: applications of molecular combing.” Brief Funct. Genomic Proteomic 1(4):385-96.) All the DNA molecules on the flow cell would be stretched in the same direction, and would be parallel to each other, and this direction would be a known, fixed direction and orientation with respect to the flow cell. This known orientation would be taken into consideration by the sequence reconstruction software (see below, and see FIG. 4). The surface of the flow cell would then bind and capture the stretched DNA molecules, in some embodiments after some cue (an added chemical reagent; a change in pH; a change in temperature, induction by light; induction by microwaves, etc.).

2. For optimum results, the flow cells used in this procedure would have their surfaces chemically modified to increase DNA binding and capture. A large literature exists on various chemical modifications useful for this purpose, as such binding and capture reactions have been used for the construction of microarrays. For example, the flow cell surface could be chemically modified using reactive groups such as aldehyde groups, amino groups, ester groups, epoxide groups, methacrylate groups, and many others (http://www.arrayit.com/Products/Microarray Slides/microarray slides.html, Lee et al. 2012, “Rapid and Facile Microwave-Assisted Surface Chemistry for Functionalized Microarray Slides”, Adv. Funct. Mater 22(4):872-878; Kwiat et al., 2012, “Non-covalent monolayer-piercing anchoring of lipophilic nucleic acids: preparation, characterization,m and sensing applications. J. Am. Chem. Soc. 134(1):280-92.

3. The stretched DNA molecules would be fragmented in situ, then amplified in situ (FIG. 3). In one embodiment, this could be carried out by “tagmentation”, such as used in the Illumina Nextera system. In this system, an in vitro transposition reaction is used to insert transposon-related sequences into long DNA molecules, thus both breaking (fragmenting) the molecules, and also added primer sequences for amplification. Once the long DNA molecules are fragmented and tagged in this way, amplification and polony formation will occur as in a normal Illumina sequencing reaction.

4. Sequencing of each polony will occur as in a normal Illumina sequencing reaction.

5. The sequence of DNA in each polony will be obtained using imaging and imaging software as in a normal Illumina sequencing reaction.

6. Custom, novel software would deconvolute the molecules on the flow cell, determining which belong to the same, original long molecule. Note that the flow cells will contain a very high density of polonies, and (unlike the drawings, FIG. 3 and FIG. 4), the polonies will not be well-separated from each other, and it will not be obvious which polonies came from the same original molecule. However, various algorithms will be capable of deconvoluting the polonies and assigning them to original long DNA molecules. There are at least two different cases for such deconvolution, one easy and one hard.

In the easy case, the genomic sequence of the DNA being sequenced is already known (this would be true if, for instance, sequencing were being done to determine haplotype). In this case, the algorithm would focus on the sequence in a particular polony, and look for other polonies “in line” (FIG. 3, FIG. 4, and see step 1 above) with the particular polony chosen, and amongst these in line polonies, search for those having sequences known to be nearby the sequence on the chosen polony. (The alternative algorithm, of identifying all polonies on the flow cell having sequences from regions spatially related to each other, then finding best-fit linear clusters, is also do-able and may be superior.)

In the hard case, an organism with a novel genomic sequence would be under study. In this case, sequence information from a related organism could be used as above, since gene orders are often similar between organisms (synteny). But even without synteny, deconvolution can be done de novo using high sequence depth (i.e., sequencing each region of the genome multiple times, such as 100 times (referred to as “100× coverage” or “100× depth”). In such a case, an algorithm would focus on a sequence from a particular polony, then find all polonies on the flow cell with at least a portion of the same sequence (for 100× coverage, there would be about 100 such colonies), then look at all “in line” sequences for all 100 polonies, and finally find in line sequences shared, and in order, by the 100 lines of polonies (FIG. 4).

Note that step 3 (fragmentation, capture by the flow cell, and sequencing) (FIG. 3) is unlikely to be 100% efficient, and therefore some or even many of the fragments from a long DNA molecule will escape sequencing. However, at high sequence coverage, missing fragments (sequence gaps) from one molecule can be filled in using sequences from another molecule on the flow cell (FIG. 4). For determining haplotypes, only linear arrays of molecules containing the distinguishing alleles of the haplotype will be useful for this purpose. There is an inter-relationship between the efficiency of step 3, and the needed sequencing depth: in cases where step 3 is less efficient (i.e., a smaller percentage of the fragments from a long molecule are sequenced), then a greater read depth is needed to compensate.

What is claimed:

1. A method for ascertaining very long regions (kilobases or tens of kilobases or more) of possibly non-contiguous DNA sequence originating on a single long molecule of DNA comprising the use of:
(i) a polony-type DNA sequencer (as defined above);
(ii) DNA combing or other method for stretching DNA molecules upon a solid support or substrate;
(iii) a support or substrate, such as a modified flow cell, that binds DNA;
(iv) a procedure for fragmenting and amplifying DNA molecules in situ (e.g. http://www.illumina.com/products/nextera_xt_dna_library_prep_kit.html, the “Nextera” method from Illumina);
(v) flow cell imaging as used on polony sequencers; and
(vi) software for using spatial, geometric or directional information from images of the flow cell, and in some cases known genomic sequences, to deconvolute polonies and reconstruct long sequences.
